(19) United States
(12) Patent Application Publication
(10) Pub. No.: US 2017/0130236 A1
(43) Pub. Date: May 11, 2017

(54) METHODS OF PLANT TRANSFORMATION USING TRANSFORMABLE CELL SUSPENSION CULTURE AND USES THEREOF

(71) Applicant: UNIVERSITY OF TENNESSEE RESEARCH FOUNDATION, KNOXVILLE, TN (US)

(72) Inventors: C. NEAL STEWART, Jr., KNOXVILLE, TN (US); JONATHAN D. WILLIS, KNOXVILLE, TN (US); JASON BURRIS, KNOXVILLE, TN (US); MITRA MAZAREI, KNOXVILLE, TN (US)

(21) Appl. No.: 15/311,199

(22) PCT Filed: Jun. 2, 2015
(86) PCT No.: PCT/US15/33753

§ 371 Date: Nov. 15, 2016

Related U.S. Application Data
(60) Provisional application No. 62/006,557, filed on Jun. 2, 2014.

(51) Int. Cl. C12N 15/82; A01H 4/00; C12N 5/00; C12Q 1/68

(52) U.S. Cl. CPC C12N 15/8205; C12Q 1/6895; A01H 4/008; C12N 5/0025; C12Q 2600/13; C12Q 2600/158

(57) ABSTRACT

The subject invention pertains to a method of rapidly transforming a plant with a gene of interest. The method of the current invention comprises the steps of a) preparing a bacterial culture, wherein the bacterial culture comprises a vector containing the gene of interest, b) producing a plant cell suspension culture from the plant, c) contacting the plant cell suspension culture with the bacterial culture to produce a plant cell transformed with the gene of interest from the plant cell suspension culture, and e) producing a plant from the plant cell transformed with the gene of interest. The method of the current invention can be designed for a high throughput transformation and screening of a plant with a plurality of genes of interest and screening the plants to identify and obtain plants having desirable characteristics.

### CROSS REFERENCE TO RELATED APPLICATION

This application claims the benefit of U.S. Provisional Application Ser. No. 62/006,557, filed Jun. 2, 2014, the disclosure of which is hereby incorporated by reference in its entirety, including all figures, tables and amino acid or nucleic acid sequences.

This invention was made with government support under 0670-2176 awarded by Advanced Research Projects Agency-Energy, U.S. Department of Energy. The government has certain rights in the invention.

### BACKGROUND OF THE INVENTION

Genetic modification of wild type plants and generation of plants with desirable characteristics is an important aspect of plant biotechnology, for example, developing of plants better suited for biofuel production. The currently available methods of plant genetic modification, for example, plant cell transformation and generation of plants therefrom, are too slow and are not suitable for high throughput screening of transformed plants with desirable characteristics.

### BRIEF SUMMARY OF THE INVENTION

The current invention provides methods for rapid production of transgenic plants and screening the transgenic plants having desirable characteristics. The method of the current invention comprises the steps of:

a) preparing a bacterial culture, for example, a culture of Agrobacterium tumefaciens strain EHA105, comprising a vector, for example, a pANIC vector, wherein the vector comprises a gene of interest,

b) producing a cell suspension culture from a plant to be transformed, optionally, via producing a callus from the plant,

c) contacting the plant cell suspension culture with the bacterial culture to produce a plant cell transformed with the gene of interest, and

d) producing a plant transformed with the gene of interest from the plant cell transformed with the gene of interest.

The transformed plants can be further screened for a desirable characteristic to identify the transformed plants of interest. The transformed plants of interest can be further propagated in to subsequent generations, for example, through seeds.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1. Comparison of the plant transformation system of the current invention with the other systems currently available.

FIG. 2. Overview of transformation methodology and workflow.

FIG. 3. ST2 transformation 7 days after inoculation with Agrobacterium pANIC10A-MYB. Black circles indicate single cells. Red circles indicate cell aggregates. Blue circles indicate non-transformed cell aggregates. Orange circles indicate transformed cell aggregates. Green circles indicate dead cells (not counted). ST2 control field shows one cell aggregate (15 cells) and two single cells. ST2 Transformed field shows one OFP-aggregate (Orange Fluorescent Protein) (14 cells) and one non-transformed aggregate 18 cells. Transformation rate=43.75%.

FIG. 4. Pre and post cryopreservation viability of suspension cell culture. Five cryopreservation experiments were performed.

FIGS. 5A-5B. For regeneration in liquid medium, 5-10 ml of culture was placed into a new flask, and liquid “REG”+Cefo 250 was added to 30 ml. Cultures were grown in shaking growth chamber. A. ST1 grown for one month in liquid REG+Cefo poured into petri plate and liquid removed. Shoots can be moved to solid media for rooting. Rooting can take about 8-10 weeks. B. Performer line 925 in Reg+Cefo Mag box after two months of being on solid media.

FIG. 6. Plants from several genotypes regenerated from suspension cultures in the greenhouse.

FIG. 7. Standard curve for lignin estimation using fluorescence.

FIG. 8. Rankings match reported switchgrass lignin results. Fluorescence emission spectra were collected from cell cultures derived from whole transgenic and control plants. Three aliquots were taken from each cell culture. SE reported. Each bar represents a cell suspension culture developed from a transgenic or wild type clone switchgrass line from either MYB or COMT constructs reported in Hui 2011 et al., and Fu 2009 et al.

FIG. 9. Clones top to bottom: #1135, #605, #925, and #770; Dates from left to right: (started on REG media at 1-30-14) Left 2-1-14, Middle 2-14-14, Right 3-4-14.

FIG. 10. Microscopy photos from epi-fluorescence microscope with: White light (top-left), GFP filters (top-right), and RFP filters (bottom).

FIG. 11. Microscopy photos from epi-fluorescence microscope with: White light (top-left), GFP filters (top-right), and RFP filters (bottom).

FIG. 12. Both plantlets with new RFP positive shoots are from the #925 clone's wild-type callus regeneration. This picture was taken 5 weeks post-transformation.

FIG. 13. Repetition 1 for #925, events: 2 (right) and 3 (left).

FIG. 14. Repetition 1 for #925, events: no event (right) and 1 (left).

FIG. 15. Repetition 2 for #605, events: 2 (right) and 1 (left).

FIG. 16. Repetition 2 for #925, events: 1 (right); Repetition 2 for #605, events: no event (left).

FIG. 17. Labeled electrophoresis gel (1% agarose) with positive bands in the 3-5 labeled wells.

FIGS. 18A-18D. An example of P605 cell suspension culture. A) 125 ml flask of cell suspension with nurse calli at the bottom of the flask. B) Micrograph of dividing P605 cell suspension. C) P605 callus recovered on filter paper from cell suspension. D) Regenerated green shoots from P605 calli.

FIG. 19. Dissimilation growth curves on Performer 605 cell culture using dual caps on each flask. Experiment was carried out in triplicate with triplicate control flasks to assess evaporation rates. The data shown represent triplicate test averages minus triplicate evaporation control averages for each time point.

FIGS. 20A-20F. The utilization of orange fluorescence to identify and monitor transgenic tissues. A and B) P605 cell suspension post-transformation empty vector control. C and D) P605 cell suspension post-transformation pANIC10A-Control. E and F) P605 cell suspension callus post transformation pANIC10A-Control. The images in top row show tissues illuminated by white light with no emission filter, whereas the images in the bottom were taken using a TxRed filter set. The photographic exposure times for each image are shown.

FIGS. 21A-21D. The utilization of orange fluorescence to identify and monitor transgenic tissues and regenerated shoots and plants. A and B) Regenerating shoots. C and D) regenerated plantlets. The images in the left column show tissues illuminated by white light with no emission filter, whereas the images in the right column using a TxRed filter set. The photographic exposure times for each image are shown.

FIGS. 22A-22B. GUS staining switchgrass cell cultures. A) Empty vector control. B) Transformed P605 cell cultures with pMDC162-PvU62.1. White arrows indicate blue GUS staining.

FIG. 23. PCR assay for the hygromycin resistance transgene DNA from cell-culture derived putative transgenic plants. Lane order 1) Molecular marker, 2) Water control, 3) Plasmid control, 4) Empty vector control, 5) Transgenic pMDC162-PvUb6, 6) Transgenic pMDC162-PvUb6, 7) Transgenic pANIC10A-Control, 8) Transgenic pMDC162-46, 9) Water. The expected amplicon size is 1 kb.

FIGS. 24A-24F. Cryopreservation of switchgrass cell cultures using FDA staining to assess viability. Viable, FDA-stained cells fluoresce green. A) Pre-cryopreservation. B) FDA-stained of cells before cryopreservation. C) Cell suspension cultures recovered 14 days post-cryopreservation. D) FDA-stained cells 14 days post-cryopreservation. E and F) Post-cryopreserved cells forming calli on filter paper.

FIG. 25. Viability of post-cryopreserved cells determined by FDA staining. At each time point, a sample was taken and measured in triplicate. Statistical analysis with SAS 9.4 for LSD>0.5 found no significant difference of viability over time.

FIG. 26. Transformation efficiencies from separate vectors and experiments.

FIG. 27. Comparison of three switchgrass transformation methods.

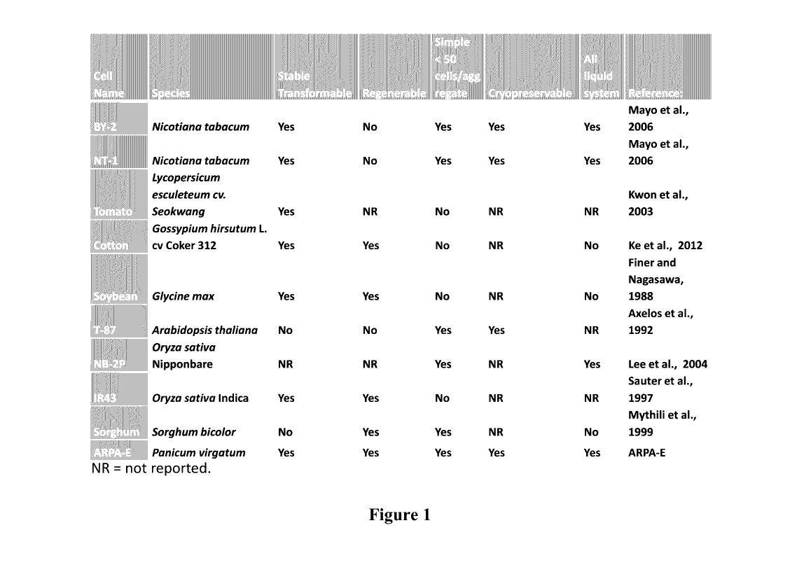

### DETAILED DISCLOSURE OF THE INVENTION

The term “about” is used in this patent application to describe some quantitative aspects of the invention, for example, concentration. It should be understood that absolute accuracy is not required with respect to those aspects for the invention to operate. When the term “about” is used to describe a quantitative aspect of the invention the relevant aspect may be varied by ±10% (e.g., ±1%, ±2%, ±3%, ±4%, ±5%, ±6%, ±7%, ±8%, ±9%, or ±10%).

The current invention provides methods for producing a transformable, non-aggregate plant cell culture that can be rapidly transformed and screened in a high throughput manner to identify and propagate plants of desirable character. Non-limiting examples of the desirable character include lower lignin contents to produce biofuels, increased oil/lipid contents and resistance to drought, heat, flooding, etc.

The methods of the current invention provide improved transformation rate compared to known techniques of plant transformation. For example, in certain embodiments, the methods of current invention provide about 5% transformation rate using embryogenic calluses and about 65% transformation rate using cell cultures. In certain other embodiments, the claimed invention provides about 1% to about 10%, about 2% to about 8%, about 3% to about 7% or about 5% transformation rate using embryogenic calluses. In further embodiments, the claimed invention provides about 30% to about 80%, about 40% to about 70%, about 50% to about 60% or about 65% transformation rate using cell culture systems.

For the purposes of the current invention, the transformation rate represents the percentage of or the ratio of number of cells transformed to the total number of cells alive at the conclusion of the step of contacting the cells to be transformed with the bacterial culture used for transformation.

The methods of the current invention comprise the steps of:

a) preparing a bacterial culture, for example, a culture of Agrobacterium spp., e.g, A. tumefaciens strain EHA105, comprising a vector, for example, a pANIC vector, wherein the vector comprises a gene of interest,

b) producing a cell suspension culture system from a plant to be transformed, optionally, via producing a callus from the plant,

c) contacting the plant cell suspension culture with the bacterial culture to produce a plant cell transformed with the gene of interest from the plant cell suspension culture, and

d) producing a plant transformed with the gene of interest from the plant cell transformed with the gene of interest.

The transformed plants can be further screened for a desirable characteristic to identify the transformed plants having the desirable characteristic. The transformed plants having the desirable characteristic can be further propagated in to subsequent generations, for example, through seeds.

For the purposes of this invention, the term “gene of interest” includes a gene that encodes for a protein or a gene that is transcribed in to an inhibitor RNA, for example, siRNA, miRNA, shRNA or RNAi, which in turn inhibits the expression of another gene.

The bacterial cultures suitable for practicing the methods of the current invention include but are not limited to Agrobacterium spp., for example, Agrobacterium tumefaciens. In one embodiment, the bacterial culture is Agrobacterium tumefaciens strain EHA105. Additional examples of bacterial cultures that can be used in the methods of the current invention are well known to a person of ordinary skill in the art and such embodiments are within the purview of the current invention. Vectors suitable for practicing the methods of the current invention include, but are not limited to, plasmids that can replicate in both E. coli, a common lab bacterium, and the bacterium, for example, Agrobacterium spp., used to insert a gene of interest into the plants. Agrobacterium based transformation vectors typically contain three key elements: plasmids selection (creating a custom circular strand of DNA), plasmids replication (so that it can be easily worked with) and T-DNA region (inserting the DNA into Agrobacterium). In certain embodiments, the vectors used in the methods of current invention are suitable for high throughput analysis of a plurality of genes.

In one embodiment, the vector is a Gateway™-compatible plant transformation vector. Certain examples of Gateway-compatible plant transformation vectors are described by Mann et al. (2012). For the purposes of the current invention, a Gateway™-compatible plant transformation vector indicates that the vector can be used according to the Gateway™ recombination cloning technology. Additional examples and aspects of the vectors suitable for use in the methods of the current invention are known to a person of ordinary skill in the art and such embodiments are within the purview of the current invention.

In an embodiment of the current invention, a culture of Agrobacterium spp. carrying a vector comprising the gene of interest is grown for about 24 hours to optical density of about 0.3 to about 0.8, about 0.4 to about 0.6 or about 0.5. Bacterial cells are then separated from the culture medium, for example, by centrifugation or filtration, and the cells are resuspended in a solution suitable for transformation.

The solution suitable for transformation of plant cells according to the methods of current invention provides necessary components to promote transformation of the plant cell with the bacteria. In an embodiment, the transformation solution contains acetosyringone which facilitates plant cell transformation. In another embodiment, the transformation solution does not contain hormones, for example, plant hormones. In a further embodiment of the invention, the bacterial culture, for example, Agrobacterium culture, is incubated in the transformation solution for about 30 min to about 2 hrs., about 45 min to about 1.5 hrs., or about 1 hr. at about 20° C. to 30° C., about 22° C. to about 28° C. or about 24° C. to about 26° C. before contacting with the plant cell suspension culture. Such incubation can further facilitate the transformation.

The cell culture suitable for transformation according to the methods of the current invention can be produced from a callus culture derived from the plant to be transformed. For example, appropriate cells from a plant of interest, for example, inflorescence meristem cells, can be developed in to calluses. These calluses can then be used in a liquid culture system to produce aggregate and non-aggregate cells suitable for transformation.

The plant cell suspension cultures can be optionally cryopreserved before the contacting step. An example of a method of cryopreservation of the plant cell suspension culture is described in the Materials and Methods section below. Additional examples of the methods of cryopreservation of plant cell suspension cultures are well known to a person of ordinary skill in the art and such embodiments are within the purview of the current invention.

Cryopreserved plant cells can be recovered, for example, by thawing in a 37° C. water bath. Thawed plant cell suspension culture can be separated from the media, for example, by centrifugation or filtration, and contacted with the bacterial culture used for transformation.

The step of contacting the plant suspension culture cells with the bacterial cells can occur over several days, for example, about 5 to about 10 days, about 6 to about 9 days, about 7 to about 8 days or about 8 days.

In one embodiment of the invention, appropriate amount of the bacterial cells are suspended in the transformation solution and optionally treated as discussed previously.

The plant cells to be transformed are separate from the media of the suspension culture or thawed cryopreserved culture, for example, by centrifugation or filtration. The plant cells are then contacted with the bacterial in the transformation solution for appropriate period of time, for example, about 24 hrs. to about 72 hrs., about 36 hrs. to about 60 hrs. or about 48 hours. In one embodiment, transformation solution is added at a ratio of 1:4 of Agrobacterium solution to cell culture and placed at room temperature on orbital shaker for about 100-150 rpm to co-cultivate for about 24 hrs. to about 72 hrs., about 36 hrs. to about 60 hrs. or about 48 hours.

After contacting the plant cells with the bacterial cells for appropriate period of time as discussed above, a bactericidal agent can be added to the mixture of plant cells and bacterial cells to kill the bacterial cells. In one embodiment, timentin (or another antibiotic) is added to the mixture to kill the bacteria. Non-limiting examples of such antibiotics include: Cefotaxime, Carbenicillin and Ampicillin.

The mixture of plant cells and bacterial cells treated with the bactericidal agent can be further incubated, optionally, in fresh media, to allow the plants cells to propagate and form calluses. Optionally, agents that stimulate callus growth are added to the plant cells. In one embodiment cefotaxime at a concentration of about 100 mg/L to about 500 mg/L, about 150 mg/L to about 400 mg/L, about 200 mg/L to about 300 mg/L or about 250 mg/L is added to stimulate callus growth. In other embodiments, proline and other amino acids can be added to stimulate callus formation. In another embodiment, the media used to further propagate calluses is liquid REG (REG=Regeneration media as reported in Li & Qu 2009). In certain embodiments, the calluses are incubated in the fresh media, optionally, in the presence of cefotaxime, on a shaker. Once the calluses grow to sufficient size, for example, about 0.5 cm to 1 cm, the calluses can be transferred to an appropriate solid media for regeneration, i.e. for development of shoots on the calluses. After appearance of shoots, the regeneration rate can be calculated by counting the number of regenerating calluses and dividing it by the total number of calluses.

Plantlets of larger than about 2-3 cm can be moved to a fresh container, for example, a Magenta box, for rooting. Rooting can occur over about 8-10 weeks. Solid media suitable for rooting in the regenerated plants are well known to a person of ordinary skill in the art and such embodiments are within the purview of the current invention. Once the root system sufficiently develops, the plants can be further grown and tested for desirable characteristics. The plants with the most desirable characteristics can be further propagated. The germplasms of the plants with desirable characteristics can be saved, for example, in the form of seeds, cell cultures, calluses, plant progenies, etc.

An embodiment of the current invention provides non-Agrobacterium-based transformation system, i.e. the transformation system where bacterium other than Agrobacterium is used to transform a plant of interest with a gene of interest.

One embodiment of the current invention provides high throughput automated handling and genome editing. In a high throughput automated method of the current invention, a plurality of plant cell cultures are transformed with a plurality of genes to produce a plurality of transformed plant cells. The plurality of transformed plants can then be screened for a desirable characteristic to identify and isolate the plants having desirable characteristics.

The methods of the current invention can be practiced in a wide variety of plants. Non-limiting examples of plants in which the current methods can be practiced include, but are not limited to, monocots and dicots such as corn (Zea mays), Brassica sp. (e.g., B. napus, B. rapa, B. juncea), particularly those Brassica species useful as sources of seed oil, alfalfa (Medicago saliva), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annum), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee Coffea spp.), coconut (Cocos nucifera), pineapple (Ananas comosus), citrus trees (Citrus spp.), cocoa (Theobroma cacao), tea (Camellia sinensis), banana (Musa spp.), avocado (Peryea americana), fig (Ficus casica), guava (Psidium guajava), mango (Mangifera indica), olive (Oleo europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena), barley (Hordeum), palm, legumes including beans and peas such as guar, locust bean, fenugreek, garden beans, cowpea, mungbean, lima bean, fava bean, lentils, chickpea, and castor, Arabidopsis, vegetables, ornamentals, grasses, conifers, crop and grain plants that provide seeds of interest, oil-seed plants, and other leguminous plants. Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pukherrima), and chrysanthemum. Conifers include, for example, pines such as loblolly pine (Pinus taeda), slash pine (Pinus elliotil), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In some embodiments, the plants (and plant cells) are corn, Arabidopsis, tobacco, soybeans, sugar cane, sorghum, cotton, canola, rice, cereals (e.g., wheat, barley, oats, rye, triticale, etc.), turf, legume forages (e.g., alfalfa and clover), pasture grasses, populus trees, switchgrass (or other biofuels) and the like. Other types of transgenic plants can also be made according to the subject invention, such as fruits, vegetables, ornamental plants, and trees.

Transformed plant cells obtained according to the methods of the subject invention can be regenerated into whole plants. Seeds produced by the transformed plants obtained according to the methods of the subject invention are also included within the scope of the subject invention. Additionally, other plant tissues and parts are included in the subject invention. The subject invention likewise includes methods of further propagating the transformed plants or cells obtained according to the methods of the subject invention. One method of producing such plants is by planting a seed of the subject invention.

Certain embodiments of the current invention also provide kits suitable for carrying out the methods of the current invention. The kit can comprise of a bacterial culture, a vector for cloning a gene of interest to be transformed in to a plant and various reagents required to produce various media required to practice the current invention.

### Materials and Methods

Plant Materials Used for Cell Culture Development

Switchgrass clones Alamo2, ST1, and ST2 were some of the clones used for switchgrass transformation. These clones were selected on the basis of performance in tissue culture and transformation efficiency. The MYB1E (ST2 background) and MYBL1 (ST1 background) were transformed with the pANIC2B-MYB construct, placed into liquid culture, and recovered (FIG. 8).

Performer clone 925 was generated by screening seed from Performer cultivar for tissue culture regeneration efficiency. ‘Performer’ switchgrass [Panicum virgatum L.] (Reg. No. CV-247, PI 644818) was cooperatively developed as a cultivar by the USDA-Agricultural Research Service and the North Carolina Agricultural Research Service, North Carolina State University, Raleigh, N.C. and released on 1 Nov. 2006.

SA1 switchgrass clone line is a cross between Alamo2 and ST1 clones.

Transformation of Switchgrass Cell Cultures

Agrobacterium strain EHA105 harboring a pANIC vector for gene transformation was grown to OD 0.5, pelleted and re-suspended in cell culture growth media with no hormones and Acetosyringone (Sigma) was added to w/v transformation solution (0.01%). Transformation solution is incubated at room temperature for 1 hour with for vir gene induction.

Cell cultures were harvested, treatment applied (filtration if applicable) and lightly centrifuged to concentrate cells. Cells to be transformed are re-suspended in induction media (growth media+no hormones+0.01% Acetosyringone) or normal growth media with hormones for control. Transformation solution is added at a ratio of 1:4 for transformation and placed at room temperature on orbital shaker for 100 rpm to co-cultivate for two days.

Timentin antibiotic is added to transformed cells to select against Agrobacterium and hygromycin antibiotic to select against non-transformed cells, respectively. Time point samples are harvested and checked before and after transformation each 24 hours for marker gene monitoring. Pictures were taken and the transformation rate was calculated by taking the number of transformed cells and dividing by the total number of cells in the picture. Transformations which were contaminated by fungal or bacterial sources (other than Agrobacterium) were discarded.

Regeneration of Switchgrass Cell Cultures:

Callus sized 0.5 to 1 cm in liquid culture were used to solid regeneration, using either REG CEFO 250 or MSB CEFO 250. After appearance of green shoots, the regeneration rate was calculated by counting the number of regenerating callus and dividing it by the total number of calluses. Plantlets of larger than 3 cm were moved to a MAG box of the same media for rooting (rooting takes approximately 8-10 weeks).

For regenerating in liquid medium, calluses of about 0.5 cm were transferred to a new flask, and REG CEFO 250 was added up to 30 ml total. When shoots were present, regeneration rate was counted by counting the number of regenerating calluses and dividing it by the total number of calluses. At this point, the shooting calluses were moved to solid REG CEFO 250 media. Either when plants grew too larger than 3 cm or when roots appeared to be longer than 1 cm, the plantlets were moved to a MAG box of the same media for rooting (approximately 8-10 weeks).

Cryopreservation of Switchgrass Cell Cultures

Protocol from Mustafa et al. (2011) was adapted to switchgrass cell cultures. First a cryogenic prep media of either 0.5 M mannitol was prepared. Growth media was added to cultures and maintained on shakers for two days. Cells were harvested by cooling in an ice bath and centrifuging for 10 min at 3000 rpm at 4° C. Supernatant was removed and replaced with a cryoprotectant solution (2M Sucrose/Maltose, 1M DMSO, 1M Glycerol and 1% L-proline). Cultures are added to pre-cooled cryotubes and placed in Nalgene's Mr. Frosty container for slow step-wise freezing for 4 hours to −80° C. Vials are then hard frozen in liquid nitrogen for 5 minutes and stored in a −80° C. freezer. Cryopreserved cells are recovered by thawing in a 37° C. water bath. Cell cultures are centrifuged washed three times with growth media and placed into multi-well plates.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Development of Switchgrass Transformable Cell Suspension Culture and Screening System for Rapid Assessment of Cell Wall Genes for Improved Biomass for Biofuel Production

Transformation and chemical characterization of plant cell wall characteristics for switchgrass is arduous and time consuming. An embodiment of the current invention provides transformable switchgrass cell culture lines with corresponding chemical fingerprinting to rapidly screen cell wall compositions in lines having modified genes.

Transgenic switchgrass plants having down-regulated lignin content, i.e. plants having down-regulated caffeic acid 3-O-methyltransferase (COMT) via inhibitor RNA or plants having overexpression of MYB4 transcription factor were used as donors for inflorescence meristem tissue to induce callus. COMT and MYB4 calluses were added to a liquid culture system to produce aggregate and non-aggregate cells to be evaluated by spectral and chemical analysis for cell wall properties.

Calluses generated from wild-type plants were used to develop a liquid culture system. Wild-type aggregate and non-aggregate cell cultures were transformed using Agrobacterium tumefaciens harboring the pANIC vector carrying the COMT inhibitor RNA or MYB4 gene and cell wall characteristics were analyzed. Transgenic COMT and MYB4 liquid cultures and transformed cultures from wild-type plants were analyzed by chemical (PyGC/MS) and spectral (FTIR/Flurolog) techniques to generate a prediction model for detecting cell wall changes. Development of this simple cell switchgrass culture can be used for developing a multiplex automatic genome engineering (MAGE) system for plants.

### Example 2—Lignin Estimation

Organosolve-extracted switchgrass lignin was used as standard control. Excitation wavelength of 300 nm to 1000 nm was used and emissions collected at 350 nm to 1050 nm. Python script was written to assimilate data and placed into a single dataset which was processed in R and later Python.

### Example 3—Genotypic Selection of Performer Line Switchgrass Clones for Efficiency in Agrobacterium-Mediated Transformation and In Vitro Culturing

Genotypes from the Performer line of switchgrass were run through a genotype pipeline to narrow down selected genotypes. Selection was based on higher transformation rates through Agrobacterium-mediated transformation as well as faster generational cycling time through tissue culture. Starting from one million seeds, 1198 that proliferated viable calli were re-cultured in vitro and cycled through regeneration with selection at each progression based on performance. Those selected for high efficiency through tissue culture were then re-cultured and transformed by Agrobacterium-mediated transformation of the invention. Upon transformation with a pporRFP positive vector (10A-GFP-Stuffer) the calli were then enumerated for transformations through RFP screening and scored. From one million seeds starting seeds, four genotypes of interest were identified.

Switchgrass is as a candidate for both forage and biofuel feedstock. It is a C4 perennial grass native to the United States. Because of the interests in agricultural and biofuel applications for switchgrass, understanding phenotypic variation and applications for genetic gain is important. A line that transforms through Agrobacterium-mediated transformation efficiently as well as cycling through tissue culture quickly was by screening genotypes of Performer line switchgrass clones to test in vitro culturing and transformation efficiency.

Primary Selection of Genotypes In Vitro

From Performer line switchgrass seed, one million seeds were germinated in vitro at 50 seeds per petri dish. Seeds were germinated on a callus induction media MP: (MS basic medium with 30 g/l maltose, 5 mg/l 2,4-D, 1 mg/l BAP, 2 g/l L-proline, 3 g/l gelzan, pH 5.8) and placed in a dark environment at room temperature. Post-germination, callus proliferation was induced and this was the basis for primary genotypic selection. Of the one million seeds, 1,198 germinated and formed viable callus. The viable callus pieces of each of the 1,198 clones were then placed on MSB media, this media type is used for the germination of somatic embryos. These were then placed in a lighted environment under T12 white lights with a 16:8 light cycle. Of the clones that successfully regenerated, shoots were counted per callus piece and enumerated as totals for each regenerated clone. The clones were then ranked by the mean number of shoots per number of calli. Those with the highest tiller counts and most healthy shoot formations were then transferred to MSO media to induce root formation. Clones were evaluated and selected at each transfer based on regeneration efficiency and quality.

Explants Taken from Regenerated Clones and Re-Cultured In Vitro

After primary selection, the best clones were removed from in vitro culture and planted into soil. Three copies of each selected genotype were then grown in the greenhouse to size and maturity to make floral meristems. These meristems were excised before emergence and put back into in vitro culture. These top nodes containing the floral meristem were surface sterilized using a sterilization solution (75% NaOH, 25% sterilized diH₂O (v/v), and 50 μl Tween 20 per 100 ml of solution). Next, the top nodes were washed thrice with sterilized diH₂O, split lengthwise and placed cut side down onto MSB media in the dark at room temperature. After three weeks the sterile inflorescence was excised, chopped, and placed on callus induction media, MP. In four to five weeks, with sub-culturing every two weeks, callus had proliferated and was evaluated. The best callus of each clone was then used for Agrobacterium-mediated transformations to get initial transformation efficiency.

Agrobacterium-Mediated Transformation of Re-Cultured Clones

The initial transformations were done with callus of each clone at the time it was of the best quality. After evaluating both callus formation and initial transformation rates, four clones were chosen as candidates for further testing. Three transformations were done with clones #605, #1135, #925 and #770. The last repetition of these transformations was unfortunately overgrown with Agrobacterium and data could not be collected. For each repetition, 100 calli (approx. 50 ml) per clone were transformed with Agrobacterium strain EHA105 with the 10A-GFP-Stuffer plasmid. This plasmid confers a gene for pporRFP allowing for visual selection of transformed callus. The culture of Agrobacterium in both repetitions were grown as one culture and only divided for the transformation and co-cultivation step to limit variation. The transformations were done according to the Noble Foundation Core Transformation Facility Protocol. After three days of co-cultivation the callus was moved to selection media MP+Tim400+Hyg70 (MP media+400 mg/l Timentin+70 mg/l Hygromycin).

Variation in Antibiotic Selection of Transformations

Some variability in the ability to be selected against antibiotic selection using Hygromycin was observed in different clones. Although clone #1135 was similar to selection of lines STI and STII, clone #770 and #925 were very strongly selected against, whereas #605 seemed healthy and even grew well as non-transformed callus (STI and STII were Alamo cultivars from the Samuel Roberts Noble Foundation). Therefore, an antibiotic selection experiment was performed. Clone #605 transformed well and it excelled in tissue culture which makes it an ideal candidate for selection. However, having escapes and overgrowth of non-transformed callus is a problem. The antibiotic selection experiment was set up using both wild-type calli and of calli that had been through transformation but had not been transformed (RFP negative). Each plate began at 1 g of callus (+/−0.005 g) and was allowed to grow in the dark for 5 weeks. The Hygromycin concentrations were a gradient of 100 mg/l, starting at 0 mg/l (control) up to 400 mg/l. Growth slowed as the concentration increased until there was no growth.

Regeneration of Preferential Genotypes

The other experiment for tissue culture efficiency was a regeneration experiment. Ten calli of each genotype were placed on REG media and replicated four times for a total of 40 calli per genotype. Every callus showed regeneration into full tillers. There was variation in time and quality of the regeneration as shown in FIG. 9. All of the four selected clones—#1135, #605, #925, and #770—had 100% regeneration on REG media.

The transgenic regeneration also showed variation amongst clones; however, to a lower extent (0-3% after 2 months on REG).

Plantlets regenerated from non-transgenic calli were transformed using the same protocol as with the transformed calli. Since Agrobacterium is a natural plant pathogen and juvenile plants are generally more susceptible to pathogens, transformation may be easier in these plantlets while having tissue set on regenerating.

PCR of Regenerated Transgenic Clones

From the selected RFP+ calli that regenerated in vitro, tissue for DNA isolation was taken. Although it is possible to amplify bacterial DNA in T0 generation transgenic plants, PCR is still a helpful tool in determining if the t-DNA is inserted. After two months only six transformations had regenerated. DNA was isolated from all six; however, only five had sufficient quality for running a PCR. The sixth would have been clone #925 from repetition 1 event 3. There was too little tissue left to re-isolate DNA from the plantlet. From the DNA of the other five regenerated transformed calli, a PCR was performed to amplify the hygromycin-transferase gene (HygT).

Growth Study of Callus Proliferation

Additional experiments were performed to characterize the efficiency in tissue culture some of the clones. A growth study was done to measure callus proliferation in selected genotypes. Approximately 0.75 g (+/−0.005 g) of sterile callus tissue was weighed and placed on a plate containing MP media and replicated three times for each genotype. These were grown for 35 days in dark and weighed after removing from the media. Since #770 was not seen as a good candidate until a later transformation it was not included with the other three candidate clones. The other three of the four top selected clones were included. This experiment facilitated comparison of the growth of the clones.

Qualitative Selection

This study was designed to find genotypes with traits favorable to genetic engineering and genetic study. The criteria were for Agrobacterium-mediated transformation and performance in tissue culture. Each step of the initial cycle of in vitro culturing served as a bottle-neck for genotypic selection. Those that germinated, proliferated in to preferable calli, and regenerated efficiently were tested further. As such, genotypes that did not meet primary criteria were removed narrowing the genotype list.

Re-Culturing of Selected Genotypes

The calli produced from the selected genotypes were evaluated for callus type. Callus type and age were evaluated. Many of the genotypes had uniform calli type yet others, such as #1135, had more variation amongst its calli. The best callus from each clone after proliferation and evaluation was then transformed through Agrobacterium-mediated transformation.

Transformation of Selected Favorable Genotypes

The calli derived from the selected clones were evaluated by the criteria of Agrobacterium-mediated transformation. For this initial transformation, efficiency was scored as either favorable or unfavorable based on its relative transformation efficiency to STI transformations by getting mean transformation efficiency per number of calli. Those having favorable traits were then used in another set of transformations consisting of the four best genotypes: #1135, #605, #925 and #770. Transformation rates were calculated at each step of the process of screening and selecting shown below in Table 1:

TABLE 1
Agrobacterium-mediated transformation of Performer clones with 10A-GFP-Stuffer EHA105

| | | | RFP+ | RFP+ | | | |
|---|---|---|---|---|---|---|---|
| | | # of | # Calli | # Calli | | | |
| | | Calli | (MP + Tim400 | (REG + Tim400 | RFP+ | # | PCR |
| | Date of | (in | Hyg70) | Hyg70) on | # Calli | Regenerated | Confirmed* |
| Clone # | transforma- | approx. | On MP 3- | REG | (REG + Tim400 | (RFP+ | (HygT |
| ID # | tion | 50 ml) | days post | Feb. 11, 2014 | Hyg70) | plantlets) | primers) |
| Rep 1 | | | At | At | At | At | Done on |
| | | | Jan. 9, 2014 | Feb. 20, 2014 | Apr. 1, 2014 | Apr. 29, 2014 | Apr. 16, 2014 |
| 1135 | Oct. 25, 2013 | 100 | 6 | 6 | 3 | 0 | 0 |
| 605 | Oct. 25, 2013 | 100 | 16 | 15 | 9 | 0 | 0 |
| 925 | Oct. 25, 2013 | 100 | 28 | 28 | 20 | 3 | 0 |
| 770 | Oct. 25, 2013 | 100 | 33 | 33 | 30 | 0 | 0 |
| Rep 2 | | | At | At | At | At | Done on |
| | | | Jan. 9, 2014 | Feb. 20, 2014 | Apr. 1, 2014 | Apr. 29, 2014 | Apr. 16, 2014 |
| 1135 | Oct. 25, 2013 | 100 | 13 | 8 | 1 | 0 | 0 |
| 605 | Oct. 25, 2013 | 100 | 21 | 21 | 11 | 2 | 2 |
| 925 | Oct. 25, 2013 | 100 | 100 | 72 | 33 | 1 | 1 |
| 770 | Oct. 25, 2013 | 100 | 100 | 80 | 70 | 0 | 0 |
| | | | A | B | C | D | C |

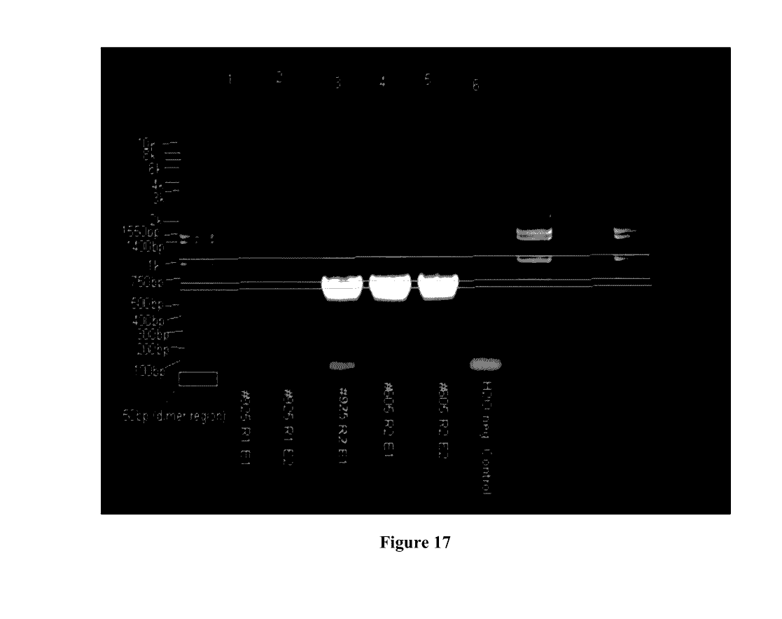

Table 1. Transformation data for each clone by repetition: from the left to right enumerations for each clone's RFP positive calli are shown in columns (A) through (C). Similarly the numbers of regenerated RFP positive shoots are in (D) and in (E) the positive results of PCR for the hygromycin-transferase gene are tabulated. (Electrophoresis gel in FIG. 17).

Variations were observed in repetitions one and two; however repetition two was higher for each clone suggesting that the variation originated from the transformation and not in the clone's genotype. To ensure limited variation, all clones were selected at the same strength of antibiotics. Variation was observed in the ability to overcome selection by each genotype.

Determining Selection Strength for Escape-Prone Selected Genotype

When using #605, selection at a higher antibiotic concentration was needed to select the transformed calli and limit escapes. A growth study using a gradient of antibiotic concentrations was used to determine the right selection concentration. Table 2 shows the growth of calli from the #605 genotype against selection by Hygromycin concentrations for five weeks:

Three repetitions were performed for each Hygromycin concentration and duplicated with both wild-type calli and calli that had gone through transformation but were not RFP+. At the Hygromycin concentration of 400 mg/l there was essentially no callus growth in the #605 genotype.

Regeneration Experiments and Observations

Regeneration of the wild-type callus in each clone was 100% as shown in the sequential pictures below. While all the calli regenerated, clone #605 was both first to show regeneration and to form shoots. Upon transfer to new media in mag boxes, #605 had the densest tillers and also the darkest green color compared to calli obtained from other clones as see in FIG. 9.

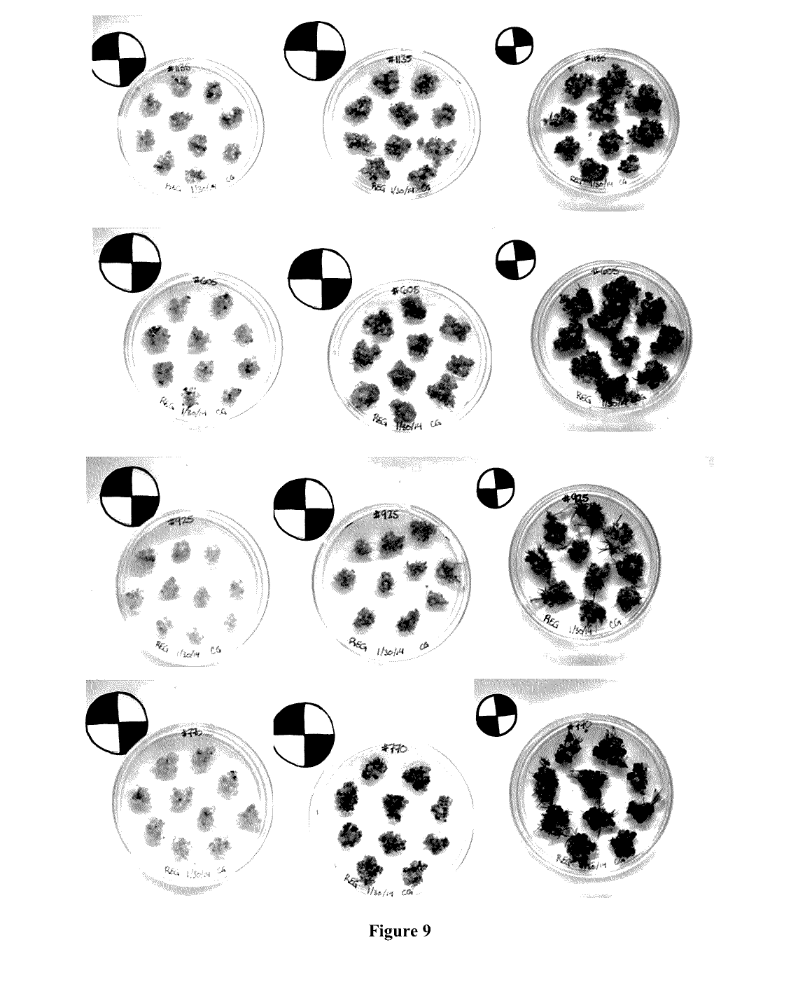

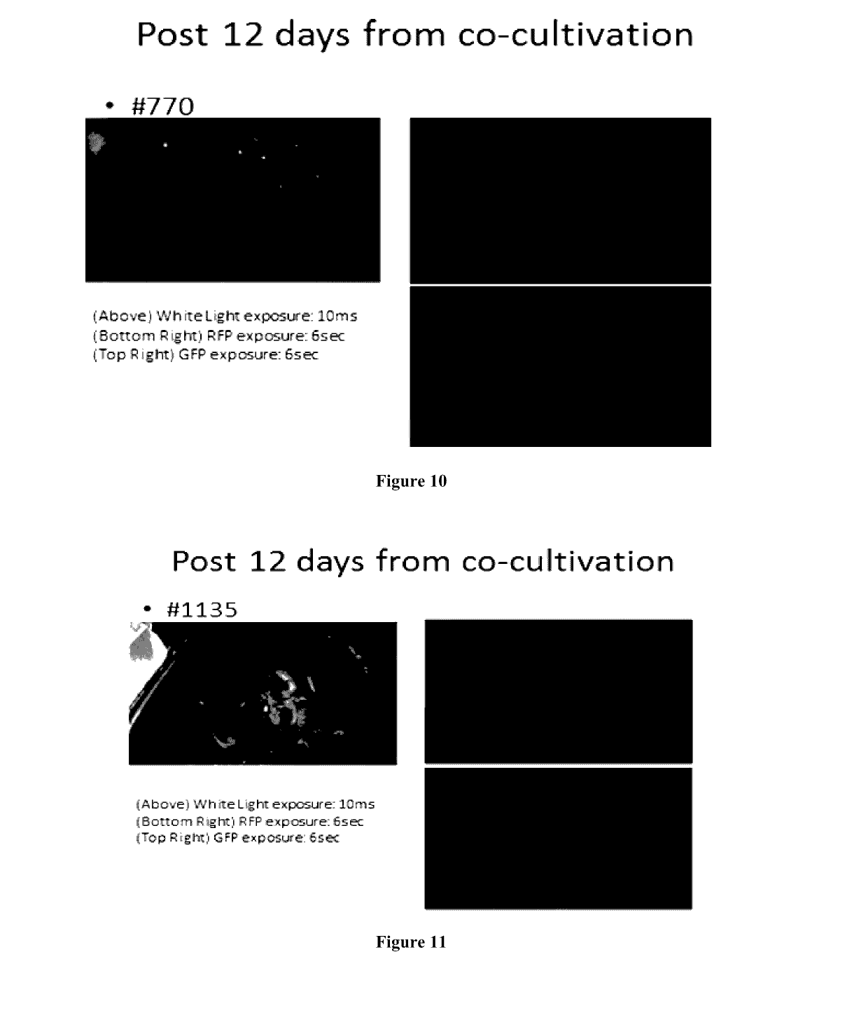

Table 1 shows a few of the RFP positive events that regenerated from any clone in both repetitions. Agrobacterium is a naturally a soil-borne pathogen. Therefore, the form of the plant most susceptible to the pathogen, for example, the newly regenerated juvenile plantlets, was transformed. After the regeneration of the wild-type calli from FIG. 9, plantlets were transformed using the same protocol from Noble's Core Transformation Facility. After twelve days there appeared to be strong RFP positive shoots while not auto-fluorescent under GFP filters using the epi-fluorescence microscope (FIG. 10).

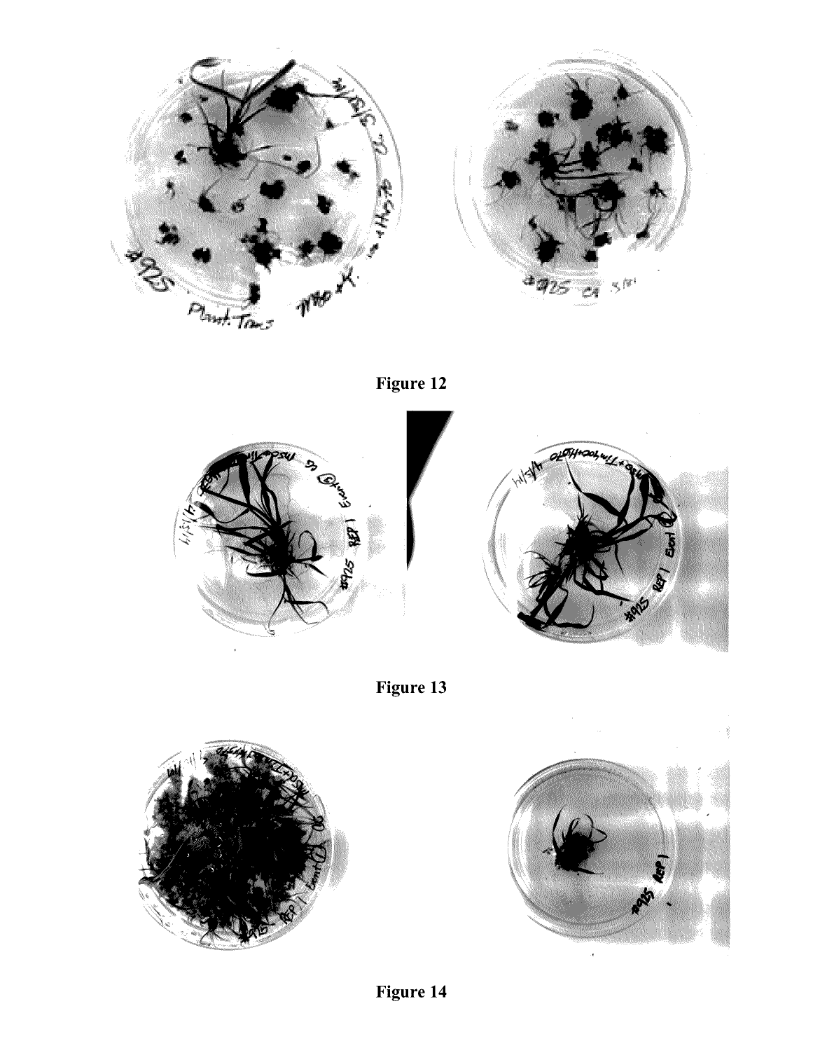

Most regenerated plantlets began to die rapidly. Therefore, the observed expression may have been transient. However by leaving the transformed plantlets on the REG media after most of them had died, a couple shoots arose that were also RFP positive and GFP negative (FIG. 12).

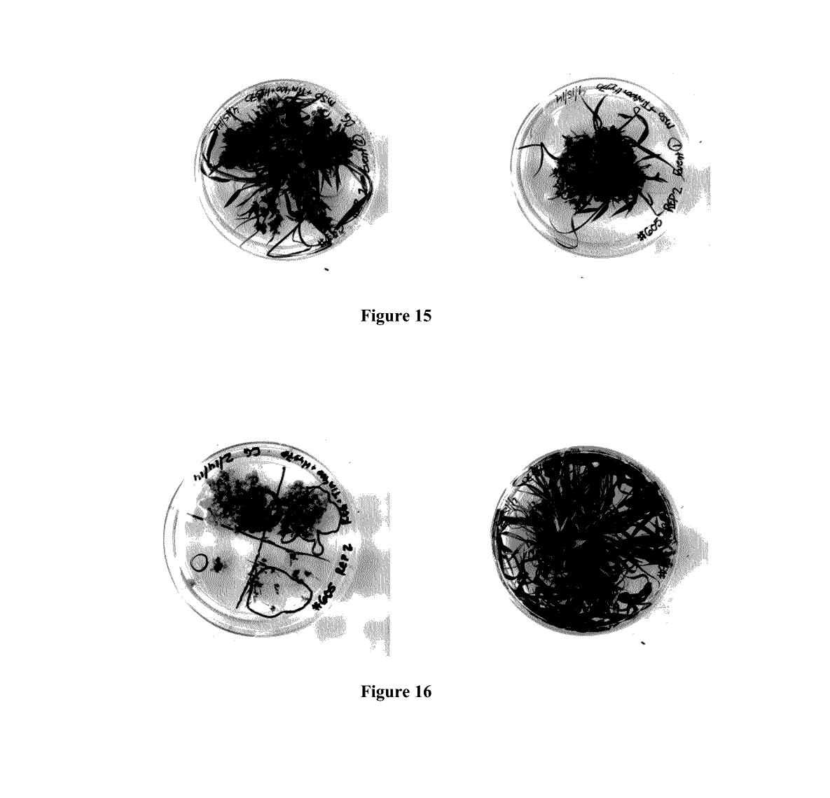

As such, eight regenerated plantlets were obtained from the callus transformations and two regenerated plantlets were obtained from the plantlet transformation. From the plantlets obtained from the callus transformation, five had adequate tissue from regeneration for DNA isolation. All of the calli transformed regenerations are shown in FIGS. 13-16.

From each possible event that regenerated (those with event numbers), DNA was isolated and used for amplification of the hygromycin-transferase (HygT) gene. The DNA from #925 Rep 1 event 3 was unable to be used and at the time of PCR (4-16-14) there was not ample tissue to excise for a re-trial of DNA isolation.

Amplification of HygT Gene in Regenerated RFP Positive Events

After excising tissue and isolating DNA from regenerated clones: #925 Rep 1 event 1, #925 Rep 1 event 2, #925 Rep 1 event 3, #605 Rep 2 event 1, #605 Rep 2 event 2, and #925 Rep 2 event 1, PCR was performed to amplify the HygT gene. Although not definitive, the PCR it serves as a better confirmation than visual screening for fluorescent proteins and selection with antibiotics alone. T₀ plants have been co-cultivated with the Agrobacterium that contain the HygT gene in their t-DNA, therefore possible amplification of the HygT gene from bacterial origin is still possible.

The amplification of the HygT gene was done with the following reaction mixture:
1. GoTaq® Green (2×) . . . @10 μl/20 μl rxn
2. (10 μM) HygT Primer-F . . . @1 μl/20 μl rxn
3. (10 μM) HygT Primer-R . . . @1 μl/20 μl rxn
4. (˜100 ng/μl) DNA Template . . . @1 μl/20 μl rxn
5. Sterile di-H₂O . . . @7 μl/20 μl rxn

Each 20 μl reaction including the H₂O control (no template and 8 μl H₂O) was placed in the thermocycler at the following cycle settings:
1. Denaturation—at 94° C. for 1 min
2. Annealing—at 60° C. for 45 sec
3. Extension—at 72° C. for 4 min
4. 50× cycles

After the PCR, products were run on a 1% agarose electrophoresis gel (250 ml TAE buffer, 2.5 g agarose, 10 μl Ethidium Bromide). The products were run at 90 watts until separation was clearly visible and the ladder had spread nearly the length of the gel. Lane order on the gel (FIG. 17) is as follows:
0. Hi-Lo Ladder
1. #925 Rep 1 event 1
2. #925 Rep 1 event 2
3. #925 Rep 2 event 1
4. #605 Rep 2 event 1
5. #605 Rep 2 event 2
6. H₂O negative control
7. Hi-Lo Ladder
8. Empty
9. Hi-Lo Ladder

As shown by the bands present in the electrophoresis gel, the events from repetition two were the only ones that tested positive for the HygT gene. It is important to mention that while the repetition one regenerated events may have not have tested positive, they may still be transgenic because the tissue obtained for PCR testing may be from a non-transgenic shoot, while some of the regeneration was still transgenic.

Callus Proliferation—Growth Study

The growth of new callus provided ample materials to characterize clones. Table 3 shows that these clones proliferated two to four times their initial weight in 35 days.

TABLE 3
Growth Study (Callus Proliferation) over 5 weeks (35 days)

| | | | | Delta | Mean |
|---|---|---|---|---|---|
| Clone # | Plate # | Initial (g) | Post (g) | (g) | (g) |
| #2-1 | P-1 | 0.7506 | 3.666 | 2.915 | |
| #2-1 | P-2 | 0.7597 | 3.087 | 2.327 | 2.640 |
| #2-1 | P-3 | 0.7511 | 3.428 | 2.677 | |
| #580 | P-1 | 0.7515 | 2.411 | 1.660 | |
| #580 | P-2 | 0.7519 | 2.571 | 1.819 | 1.739 |
| #580 | P-3 | 0.7559 | (contaminated) | n/a | |
| #605 | P-1 | 0.7506 | 3.89 | 3.139 | |
| #605 | P-2 | 0.7579 | 3.834 | 3.076 | 3.103 |
| #605 | P-3 | 0.7584 | 3.853 | 3.095 | |
| #751 | P-1 | 0.7550 | 3.200 | 2.445 | |
| #751 | P-2 | 0.7602 | 3.042 | 2.282 | 2.651 |
| #751 | P-3 | 0.7540 | 3.981 | 3.227 | |
| #801 | P-1 | 0.7524 | 2.899 | 2.147 | |
| #801 | P-2 | 0.7567 | 3.097 | 2.340 | 2.292 |
| #801 | P-3 | 0.7571 | 3.145 | 2.388 | |
| #817 | P-1 | 0.7555 | 2.580 | 1.825 | |
| #817 | P-2 | 0.7561 | 2.295 | 1.539 | 1.728 |
| #817 | P-3 | 0.7545 | 2.576 | 1.822 | |
| #925 | P-1 | 0.7544 | 3.104 | 2.350 | |
| #925 | P-2 | 0.7547 | 2.720 | 1.960 | 2.185 |
| #925 | P-3 | 0.7553 | 3.001 | 2.246 | |
| #1135 | P-1 | 0.7556 | 3.674 | 2.918 | |
| #1135 | P-2 | 0.7527 | 3.297 | 2.538 | 2.675 |
| #1135 | P-3 | 0.7592 | 3.327 | 2.678 | |

Clone #605 had the fastest proliferating calli and grew four times its initial weight. Clone #770 was not seen as a valuable candidate until a later transformation. The other three of the top performing clones were seen as strong proliferators as well as having other desirable traits. Clones were grown on MP+Tim400 media (MP media supplemented with Timentin 400 mg/l) to prevent contamination because there was no sub-culturing during this experiment. All of the preferable genotypes nearly tripled in calli weight and therefore are not limited in potential by growth in vitro.

Conclusion

This example of the current invention provides transgenic switchgrass having improved genotypes. The improved genotypes are more efficient in generational cycling through tissue culture while also responding to in vitro culturing more favorably. These clones are also more susceptible to Agrobacterium-mediated transformation.

The high amount of variation in the transformation protocols being used as well as the results from even standardized transformations currently used is undesirable. Regeneration from a genotype can quickly be evaluated from wild-type callus; however regeneration from callus post-transformation still lags behind. Transformations are only valuable when they can be regenerated into full plants.

The invention provides the clones #605, #925, and #770 which outperformed the rest of the one million genotypes tested. Clone #605 is a strong transformer, efficient in tissue culture and requires more antibiotic selection. A higher resistant genotype would be able to be slowly selected without killing any events with too much antibiotics before the gene is strongly expressed.

Clone #925 (also referred to as P925 provided herein can be easily selected while also being easily transformed.

Clone #770 (also referred to as P770) is similar to #925; however, it is extremely sensitive to selection.

### Example 4—Generating Plants and Suspension Cultures from Clone #605

Clone #605 obtained from the Performer Cultivar as described in Example 3 is hereinafter identified as P605. P605 plants recovered from tissue culture were maintained in the UT Racheff greenhouse in 3 gallon pots under 16 hour days. Explants for tissue cultures were taken from greenhouse grown tiller meristems, sterilized in 20% bleach, rinsed 3 times with sterile water. In a sterile laminar hood, washed tillers were split longitudinally with a scalpel blade and placed cut side down into Petri plates with MSB media. Plates were incubated for two weeks at room temperature in the dark to induce fresh inflorescences. After sufficient production, inflorescences were cut into 1 cm sections and placed onto callus induction MP media. Callus was sub-cultured each 2-3 weeks moving to fresh media and removing from culture any browning or dying tissues. Type 2 callus (white to slightly yellow, friable) was chosen exclusively for subculture. Type 2 callus is cultured for 3-4 months and then used for transformation or establishment of suspension cultures.

Initiation of Cell Suspension Cultures

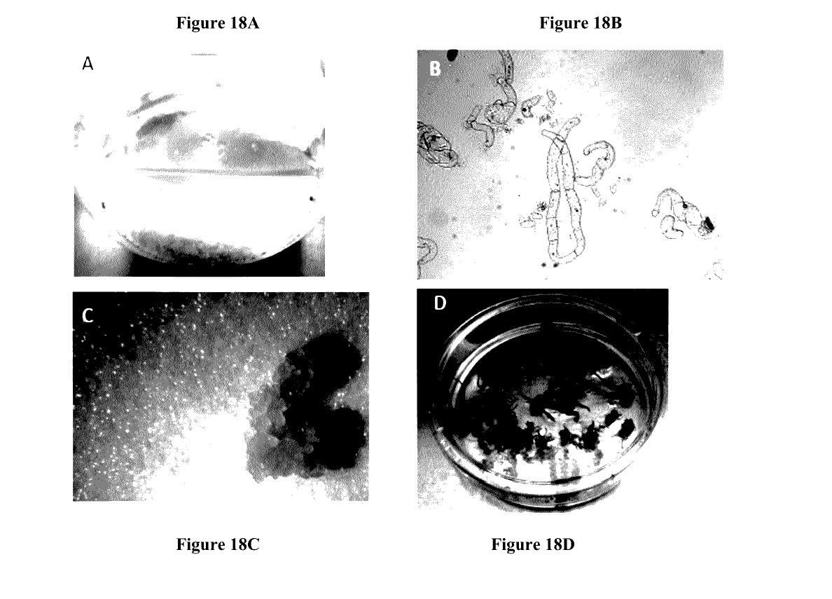

Approximately one gram (fresh weight) of freshly cultured Type 2 callus was placed into a 125 ml flask with a silicone vented top with 30 ml plant cell growth medium (MSO+maltose+0.44 μm BAP+9 μM 2,4-D) was used to initiate cell suspension cultures as described previously. The flask was shaken in the dark at room temperature at 100 rpm. Cultures were replenished at 2-week intervals by removing 8-10 ml of media and replacing it with 8-10 ml of fresh media (FIG. 18). After 3-4 months of initiating liquid cell culture, a heterogeneous mixture of suspension cultures, including free cells, was established. The culture was then grown in larger flasks (500 ml to 1000 ml) that contained 150 ml or 300 ml of media.

Cell Culture Viability, Growth Rate and Enclosure Comparison

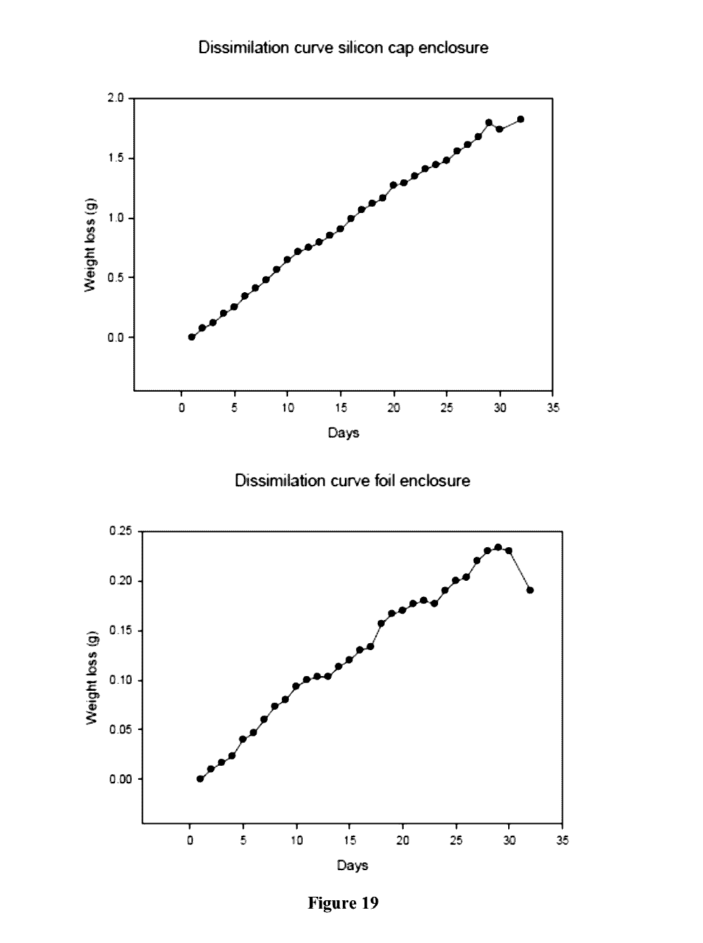

Once established, cell cultures were characterized for viability and growth. Viability was estimated using fluorescein diacetate (FDA) to measure live-cell growth. Viable FDA-stained cells are green-fluorescent. A growth curve was determined using a dissimilation curve method modified slightly from Schripsema et al. For the dissimilation curve, cell culture inoculum of equal to 10 ml of fresh cells from the same flask were added to triplicate flasks with silicon cap or foil enclosures and 40 ml of media was added to each flask. Triplicate control flasks with corresponding enclosures with media but no cells were used to measure evaporative losses. All flasks were measured at the same time, on the same scale at each time point. Time point collection began at initiation and each 24 hours for 30 days. Additional time points at days 32 and 42 were also collected. Weight loss of the experimental flasks was interpreted as growth of cell cultures. Weight loss from evaporation control flasks was removed from experimental flasks. Means of triplicates are reported in FIG. 19.

Stable Agrobacterium Transformation:

A. tumefaciens strains EHA105 harboring a transformation vector of pANIC10A-Control, pANIC17B-ZmUbi or pMDC162-promoter were freshly streaked onto YEP-Rif₅₀-Kan₅₀ agar media from a frozen glycerol stock and placed into 28° C. and incubated for two days. A starter culture was made with 2 ml liquid YEP-Rif₅₀-Kan₅₀ and inoculated with a single colony and grown overnight with shaking at 250 rpm. A transformation culture was prepared by inoculated 100 ml of liquid YEP-Rif₅₀-Kan₅₀ with 100 μl starter culture and grown overnight. The Agrobacterium culture was centrifuged at 5,000 rcf for 15 min to pellet cells, which were then resuspended to a final 0.5 OD₆₀₀ with liquid growth media. The Agrobacterium culture was induced with fresh acetosyringone (Sigma) added to a final concentration of 100 μM.

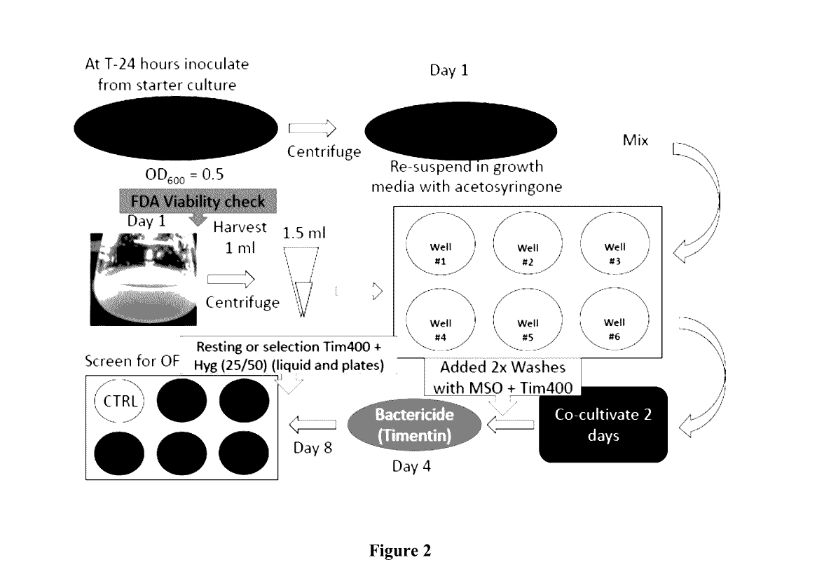

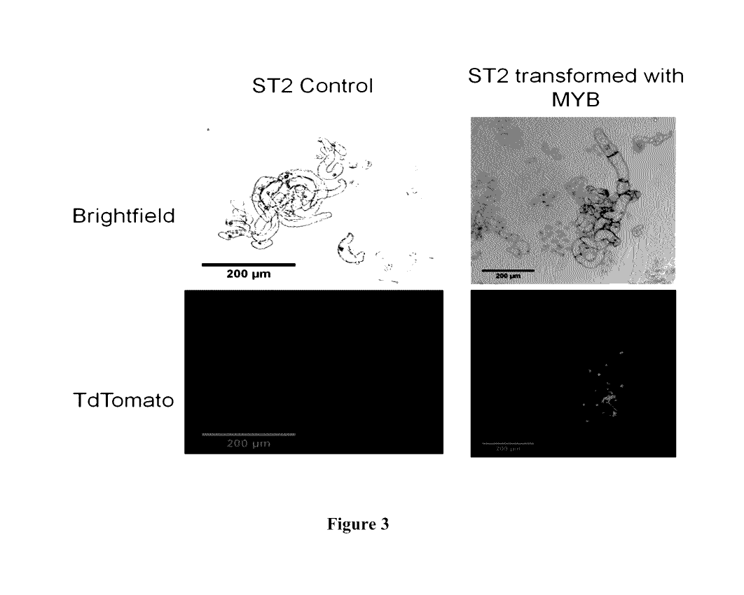

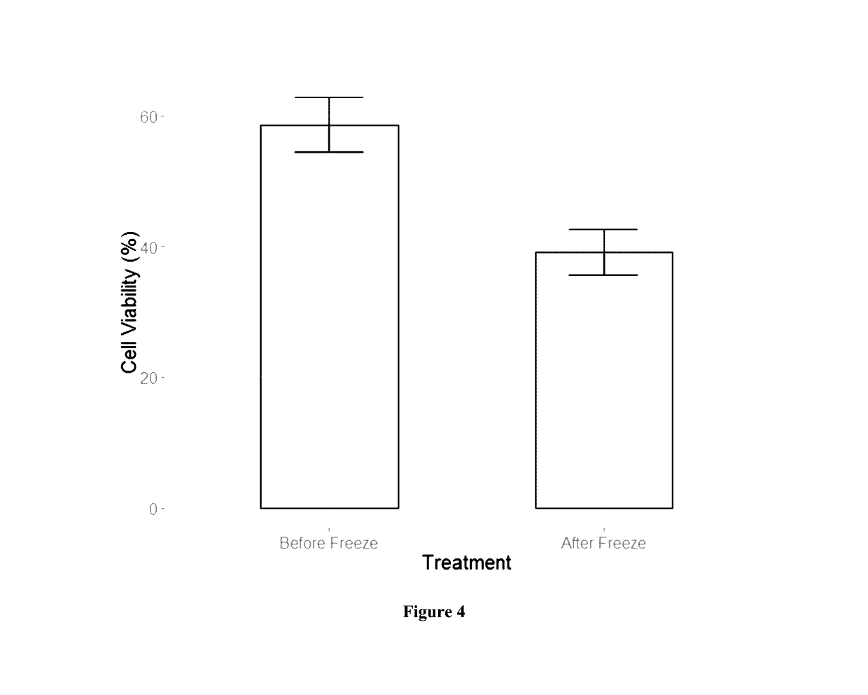

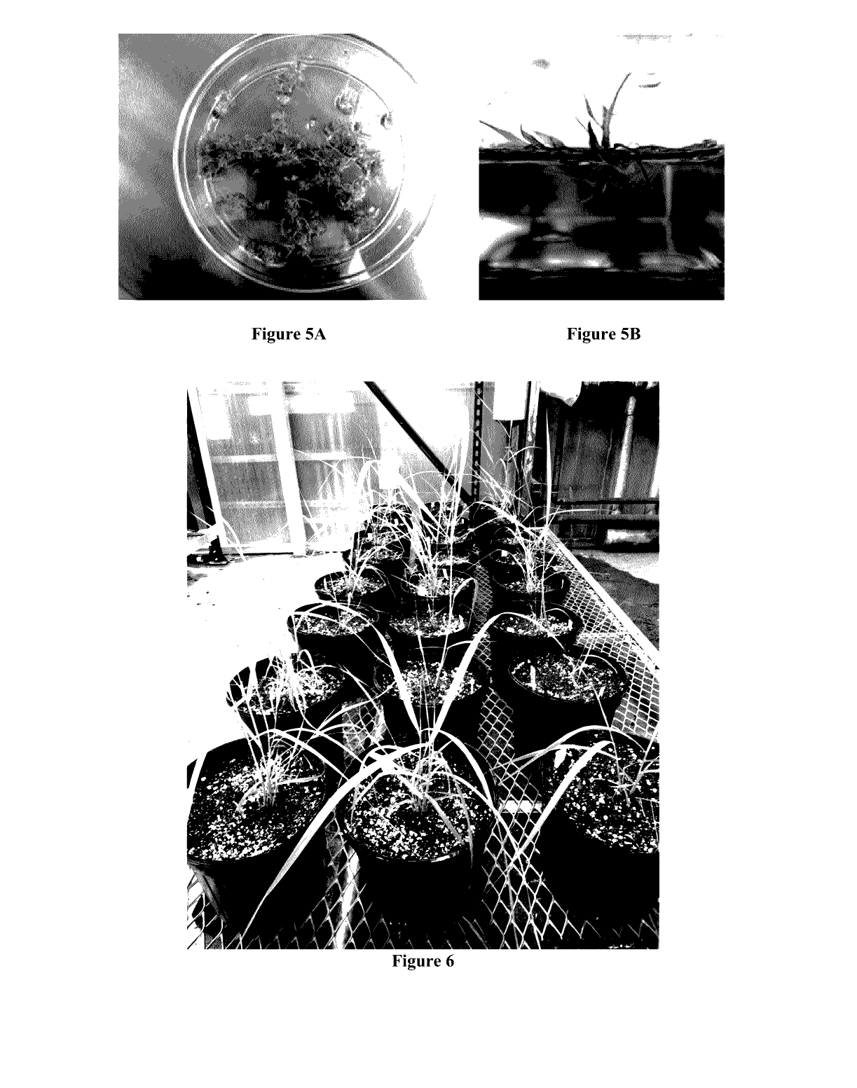

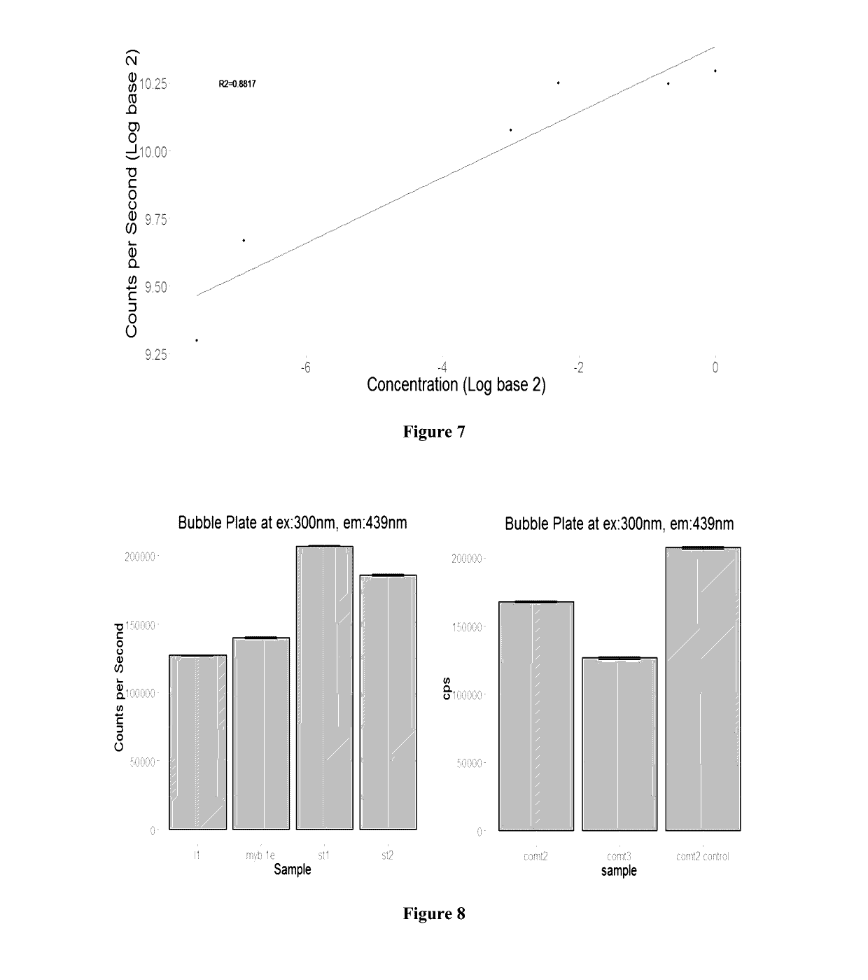

Switchgrass suspension culture aliquots were centrifuged at 4,000 rcf for 10 min to collect the cells. Switchgrass cells were co-incubated with Agrobacterium on 6-well plates for 48 h. The plates were sealed with double layer of surgical tape and placed back on a shaker at 100 rpm (FIG. 2). After co-cultivation, switchgrass cells were pipetted from the plate, centrifuged at 100 rcf and washed twice with growth media plus Timentin (400 mg/L). Cell cultures were maintained for two weeks in growth media with Timentin followed by for selection of transgenic cells in media containing hygromycin at 25 mg/L for 4 weeks and further followed by selection in media containing hygromycin at 50 mg/L for 6 weeks (FIG. 2).

Characterization of Transformed Cell Cultures:

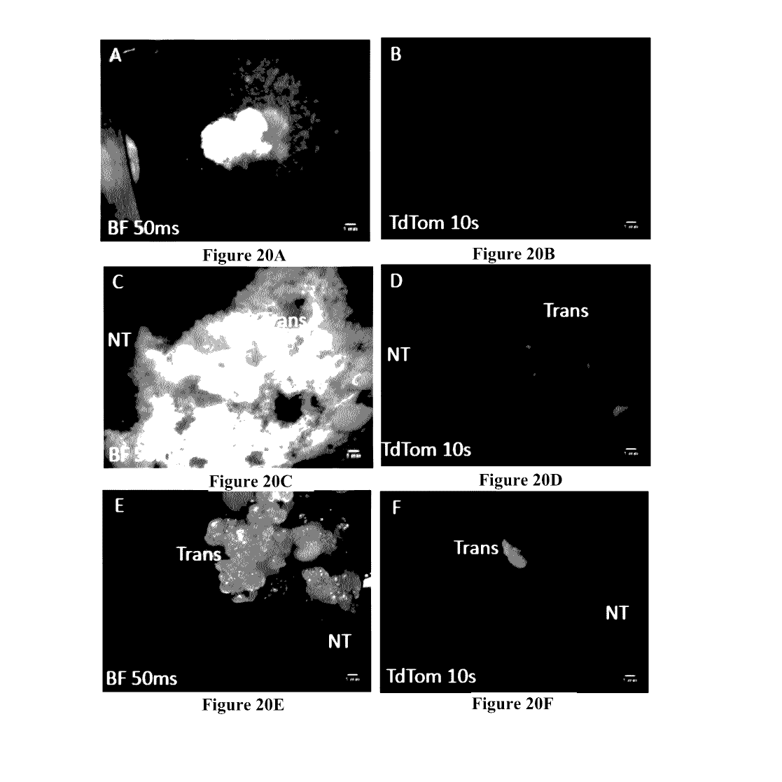

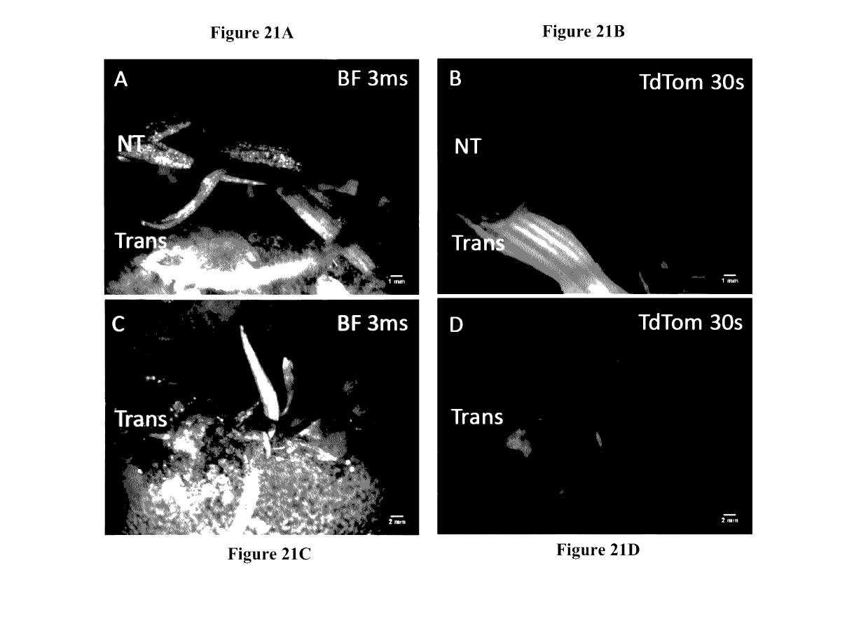

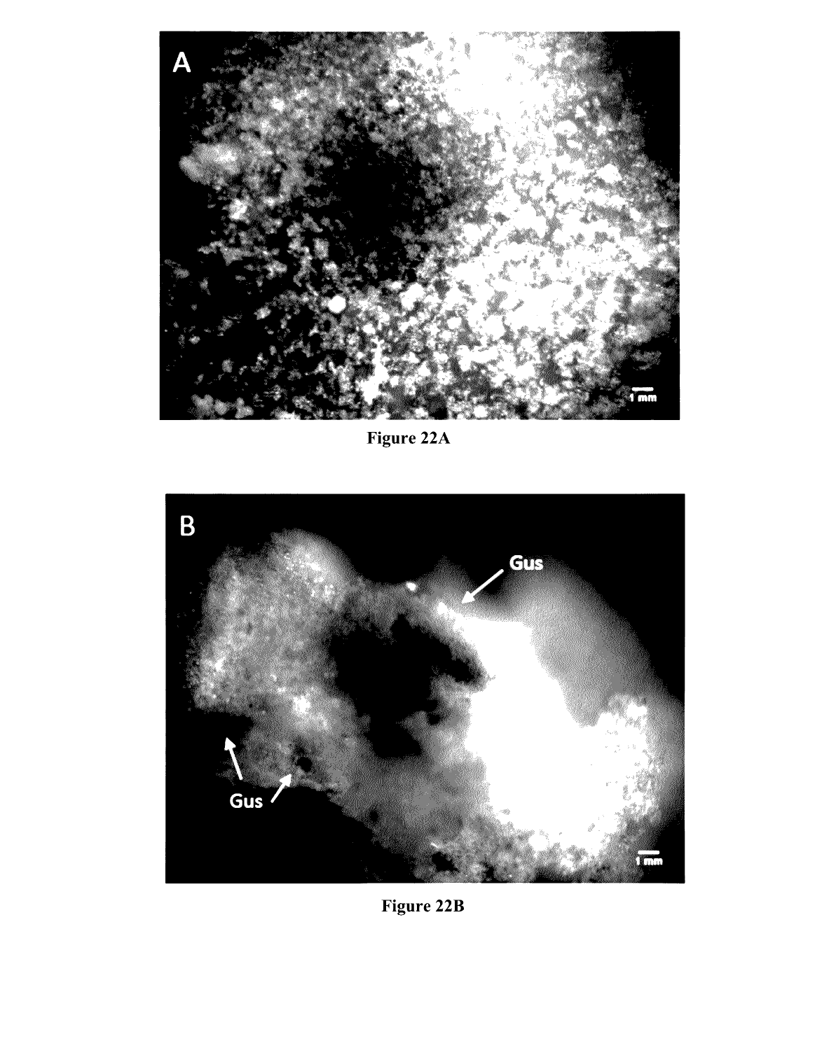

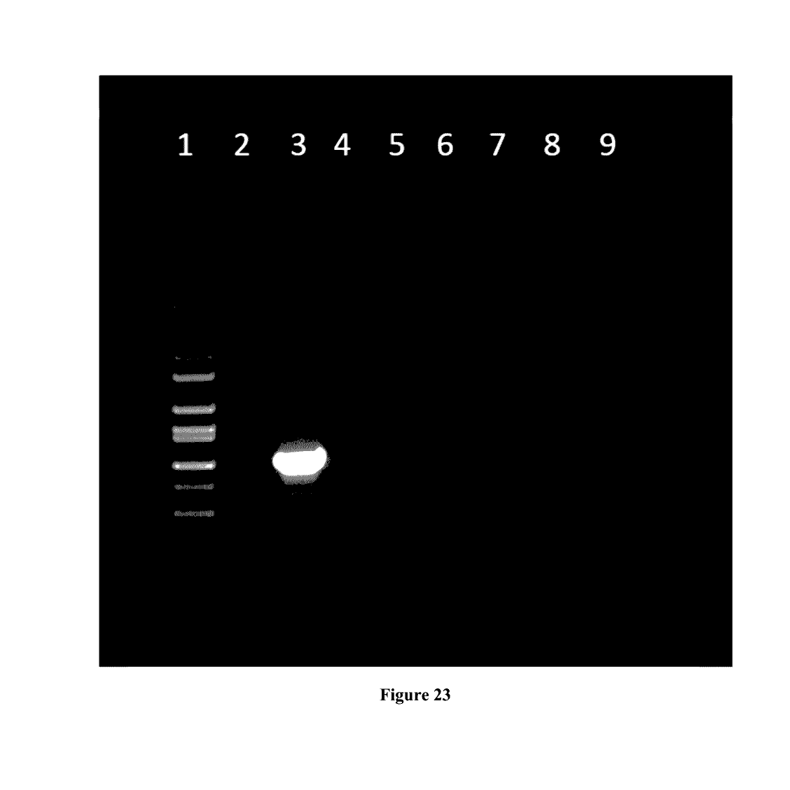

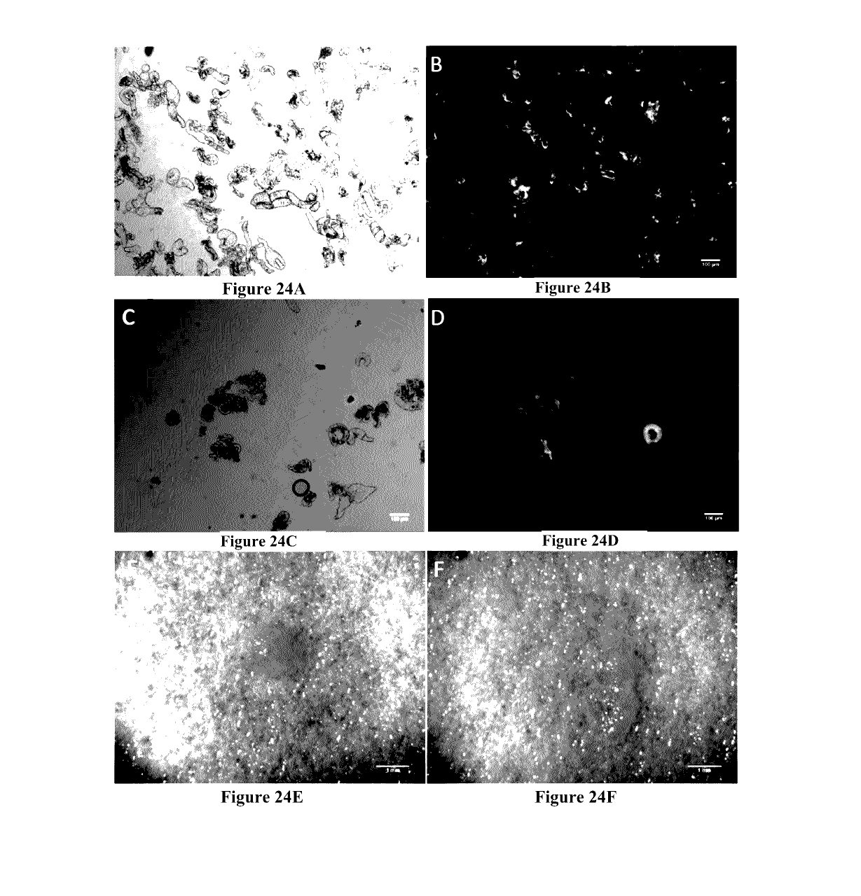

Transformed cell cultures that survived hygromycin selection could be effectively monitored by use of expression of the orange fluorescent protein through regeneration of plantlets (FIGS. 20 and 21). GUS staining also was useful to assess whether cells were transgenic (FIG. 23) using X-gluc (1 mg/ml) in 50 mM KPO₄ buffer with 0.1% Triton-X100 and 10% DMSO to liquid cell culture plates with transformed cell populations. Stained cells were incubated overnight at 37° C. for activation followed by washing with 70% ethanol. Characterization was performed by PCR confirmation of T-DNA insert and vector-backbone components from genomic DNA extracted from green shoots.

Regeneration of Whole Plants:

Shoots were regenerated from calli using REG+Cefotaxime 250 (mg/L) media for 2-3 weeks and transferred into Magenta GA7 boxes on MSO media for rooting. The regeneration efficiency was 52.6% (Table 4). Rooted plants were moved to 2 inch pots with Farfard 3B soil mix under humidity domes for acclimation.

TABLE 4
The regeneration efficiency of various transgenic lines.

| | | | Total | |
|---|---|---|---|---|
| | Regeneration | Green | calli | |
| Construct | date | shooted calli | plated | Construct |
| pMDC162-OsU6 | Apr. 23, 2015 | 7 | 21 | |
| pMDC162-PvUbi2 | Apr. 23, 2015 | 10 | 18 | |
| pMDC162-PvU62.1 | Apr. 23, 2015 | 10 | 15 | |
| pMDC162-PvU62.2 | Apr. 23, 2015 | 11 | 20 | |
| pANIC17B-OsU6 | May 5, 2015 | 8 | 12 | |
| pANIC17B-2X35s | May 5, 2015 | 4 | 12 | |
| pANIC10A-TcEG1 | May 5, 2015 | 11 | 18 | |
| Total | | 61 | 116 | 52.6% |

Cryopreservation and Recovery of Cell Cultures:

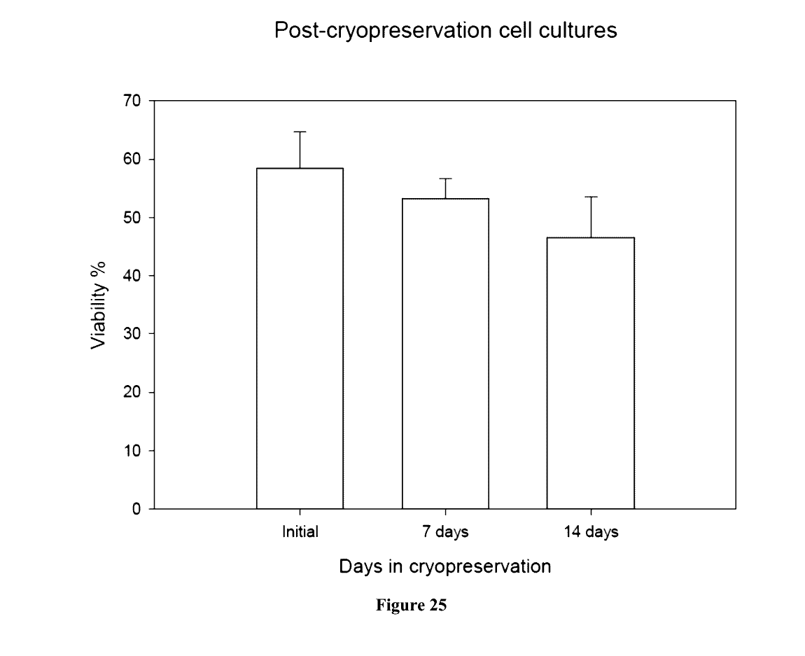

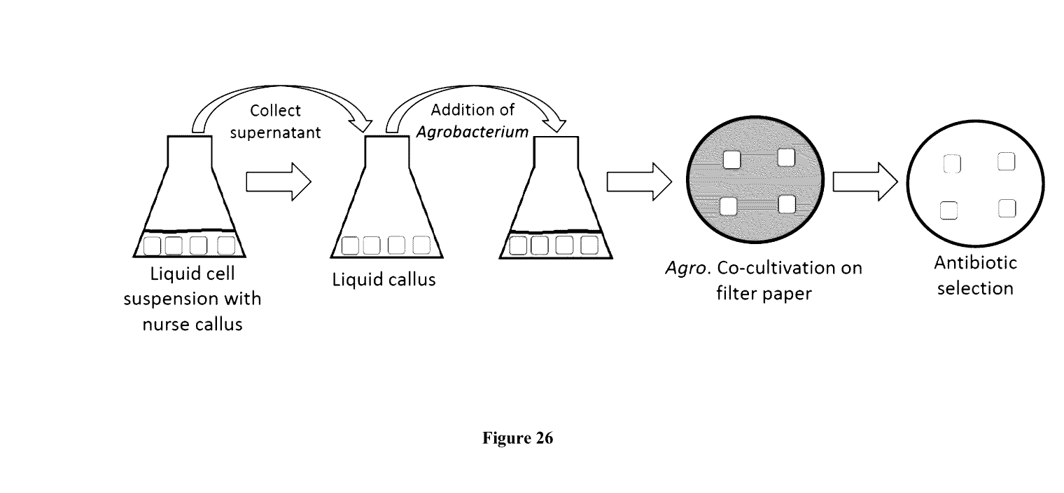

Cryopreservation of cell cultures is a desirable means of storing viable cells of effective genotypes for distribution and use, given the lengthy time, lasting for several months, and efforts needed to produce switchgrass cell cultures. Cryopreservation was performed using a protocol modified from Mustafa et al. Actively growing cell cultures were pretreated with a cryogenic prep media (MSO+maltose+0.44 μm BAP+9 μM 2,4-D+0.5 M mannitol) for 48 h in shaker flasks. Following pretreatment, the culture flasks were placed on ice for 60 min shaking at 40 rpm and then centrifuged for 10 min at 3000 rpm and 4° C. The supernatant was removed and replaced with a cryoprotectant solution (2 M sucrose, 1 M DMSO, 1 M glycerol and 1% L-proline.). The cultures were shaken on ice for 60 min. Aliquots of ˜1.8 mL of cells in the cryoprotectant solution were transferred to pre-cooled cryotubes (from −20° C. freezer) and placed in a Nalgene's Mr. Frosty container filled with 250 mL isopropanol at 4° C. The containers were placed in a −80° C. freezer for 4 h for a slow, step-wise cooling. Cell culture cryovials were then flash frozen in liquid nitrogen for 5 min for the final freezing treatment and subsequently returned to −80° C. freezer for storage. When cell recovery was desired, the cryopreserved cells were submersed in a heated water bath at 37° C. till complete thawing had occurred (about 5 minutes). Once thawed the tubes were centrifuged for 5 min at 3000 rcf. The cells were then placed onto growth media or onto sterile filter paper above the media. A small portion of the cells were collected and used for FDA viability staining (FIG. 25). While there is an apparent decrease in viability with cryopreservation time, the difference was not statistically significant. Viability post-cryopreservation ranged from 45% to 58% (FIG. 26).

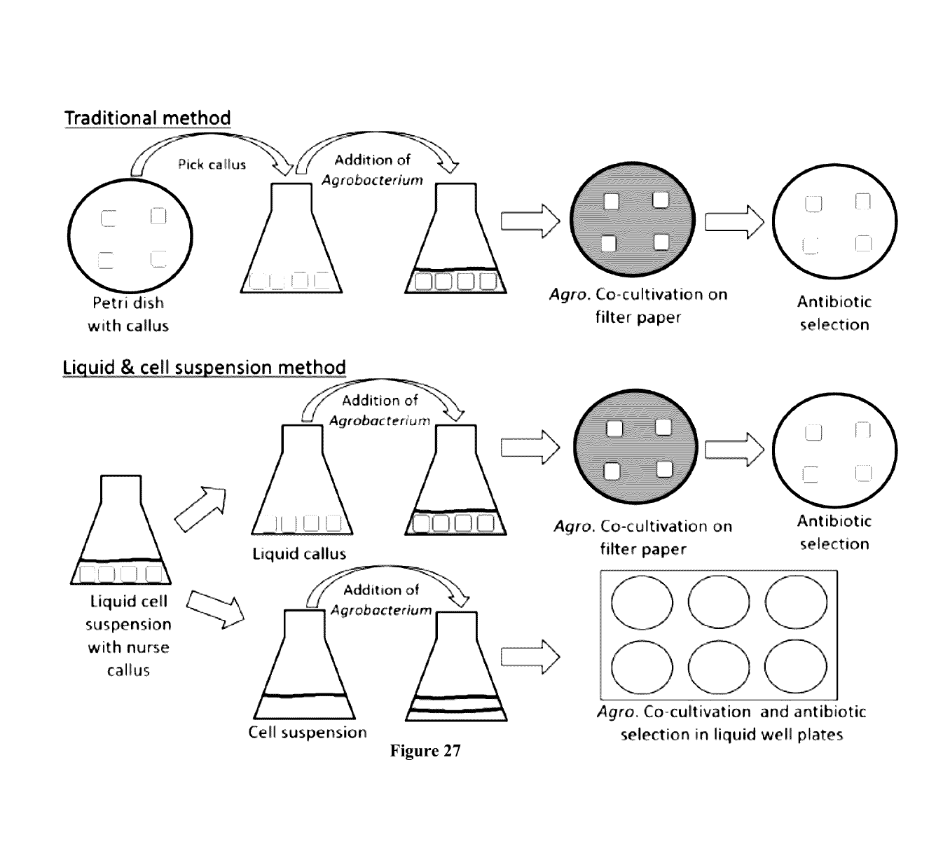

The cell culture-based technique enables switchgrass to be transformed efficiently in less time than established methods (FIG. 27). The cells are crypopreservable and regenerable. This Example provides the practice of the current invention on Performer 605 clonal switchgrass line; however, a person of ordinary skill in the art can appreciate that the invention can be applied to other switchgrass germplasms and also other plants.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Axelos, M., Curie, C., Mazzolini, L., Bardet, C., and Lescure, B. (1992). A protocol for transient gene-expression in Arabidopsis thaliana protoplasts isolated from cell-suspension cultures. Plant Physiol Bioch 30, 123-128.
2. Finer, J. J., and Nagasawa, A. (1988). Development of an embryogenic suspension-culture of Soybean (Glycine max Merrill). Plant Cell Tiss Org 15, 125-136.
3. Ke, L., Liu, R., Chu, B., Yu, X., Sun, J., Jones, B., Pan, G., Cheng, X., Wang, H., Zhu, S., et al. (2012). Cell suspension culture-mediated incorporation of the rice bel gene into transgenic cotton. PloS one 7, e39974.
4. Kwon, T. H., Kim, Y. S., Lee, J. H., and Yang, M. S. (2003). Production and secretion of biologically active human granulocyte-macrophage colony stimulating factor in transgenic tomato suspension cultures. Biotechnology letters 25, 1571-1574.
5. Lee, T. J., Shultz, R. W., Hanley-Bowdoin, L., and Thompson, W. F. (2004). Establishment of rapidly proliferating rice cell suspension culture and its characterization by fluorescence-activated cell sorting analysis. Plant Mol Biol Rep 22, 259-267.
6. Mann, D. G. J., P. R. LaFayette, L. L. Abercrombie, Z. R. King, M. Mazarei, M. C. Halter, C. R. Poovaiah, H. Baxter, H. Shen, R. A. Dixon, W. A. Parrott, C. N. Stewart, Jr. (2012). Gateway-compatible vectors for high-throughput gene functional analysis in switchgrass (Panicum virgatum L.) and other monocot species. Plant Biotechnology Journal 10:226-236.
7. Mayo, K. J., Gonzales, B. J., and Mason, H. S. (2006). Genetic transformation of tobacco NT1 cells with Agrobacterium tumefaciens. Nature protocols 1, 1105-1111.
8. Mustafa, N. R., de Winter, W., van Iren, F., and Verpoorte, R. (2011). Initiation, growth and cryopreservation of plant cell suspension cultures. Nature protocols 6, 715-742.
9. Mythili, P. K., Seetharama, N., and Reddy, V. D. (1999). Plant regeneration from embryogenic cell suspension cultures of wild sorghum (Sorghum dimidiatum Stapf.). Plant Cell Rep 18, 424-428.
10. Sauter, M. (1997). Differential expression of a CAK (cdc2-activating kinase)-like protein kinase, cyclins and cdc2 genes from rice during the cell cycle and in response to gibberellin. Plant J 11, 181-190.
11. Parrish, David J., and John H. Fike. “The biology and agronomy of switchgrass for biofuels.” BPTS 24, no. 5-6 (2005): 423-459.
12. Schmer, Marty R., Kenneth P. Vogel, Robert B. Mitchell, and Richard K. Perrin. “Net energy of cellulosic ethanol from switchgrass.” Proceedings of the National Academy of Sciences 105, no. 2 (2008): 464-469.
13. Li, Ruyu, and Rongda Qu. “High throughput Agrobacterium-mediated switchgrass transformation.” Biomass and Bioenergy 35, no. 3 (2011): 1046-1054.
14. Murashige & Skoog 1965, (physiol. Plant. 15:473-497)
15. Gamborg et al., 1968, (Expl. Cell. Res. 50:150-158)
16. Hoekema, A., P. R. Hirsch, P. J. J. Hooykaas, and R. A. Schilperoort. “A binary plant vector strategy based on separation of vir- and T-region of the Agrobacterium tumefaciens Ti-plasmid.” (1983): 179-180.
17. Burris, J. N., Mann, D. G. J., Joyce, B. L. and Stewart, C. N. (2009) An improved tissue culture system for embryogenic callus production and plant regeneration in switchgrass (Panicum virgatum L.). Bioenerg Res 2, 267-274.
18. Li, R. Y. and Qu, R. D. (2011) High throughput Agrobacterium-mediated switchgrass transformation. Biomass Bioenerg 35, 1046-1054.
19. Mazarei, M., Al-Ahmad, H., Rudis, M. R. and Stewart, C. N., Jr. (2008) Protoplast isolation and transient gene expression in switchgrass, Panicum virgatum L. Biotechnology journal 3, 354-359.
20. Mustafa, N. R., de Winter, W., van Iren, F. and Verpoorte, R. (2011) Initiation, growth and cryopreservation of plant cell suspension cultures. Nature protocols 6, 715-742.
21. Schripsema, J., Meijer, A. H., Vaniren, F., Tenhoopen, H. J. G. and Verpoorte, R. (1990) Dissimilation curves as a simple method for the characterization of growth of plant-cell suspension-cultures. Plant Cell Tiss Org 22, 55-64.

We claim:

1. A method of transforming a plant with a gene of interest, the method comprising the steps of:
a) preparing a bacterial culture, wherein the bacterial culture comprises a vector containing the gene of interest,
b) producing a plant cell suspension culture from the plant,
c) contacting the plant cell suspension culture with the bacterial culture to produce a plant cell transformed with the gene of interest from the plant cell suspension culture, and
d) producing a plant from the plant cell transformed with the gene of interest.

2. The method of claim 1, wherein the bacterial culture is Agrobacterium spp.

3. The method of claim 2, wherein the Agrobacterium spp. is Agrobacterium tumefaciens strain EHA105.

4. The method of claim 1, wherein the vector is a pANIC vector.

5. The method of claim 1, wherein the step of producing the cell suspension culture from the plant to be transformed comprises producing a callus from the plant and culturing the callus to produce the plant cell suspension culture.

6. The method of claim 1, wherein the step of contacting the cell suspension culture with the bacterial culture is performed in presence of a transformation solution, wherein the transformation solution comprises an agent that facilitates the transformation.

7. The method of claim 6, wherein the agent that facilitates the transformation is acetosyringone.

8. The method of claim 1, wherein the step of producing the plant transformed with the gene of interest from the plant cell transformed with the gene of interest comprises the steps of:
a) culturing the plant cell transformed with the gene of interest to produce a callus of the cells transformed with the gene of interest,
b) regenerating the callus to grow shoots, and
c) rooting the callus comprising the shoots to produce the plant transformed with the gene of interest.

9. The method of claim 8, wherein culturing the plant cell transformed with the gene of interest to produce the callus of cells is performed in the presence of an agent that promotes the cell transformed with the gene of interest to produce the callus.

10. The method of claim 9, wherein the agent that promotes the cell to produce the callus is cefotaxime.

11. The method of claim 8, the method further comprising testing the plant for a desirable characteristics.

12. The method of claim 1, the method further comprises collecting seeds from the plant transformed with the gene of interest.

13. The method of claim 12, the method further comprises germinating the seeds to produce progeny of the plant transformed with the gene of interest.

14. The method of claim 1, wherein the plant cell suspension culture produced in step b) can be optionally cryopreserved before the contacting step c).

15. A method of obtaining a germplasm from a plurality of germplasms, the germplasm having a higher transformation rate compared to the other germplasms in the plurality of germplasms, the method comprising the steps of:
a) incubating the plurality of germplasms under conditions to induce proliferation of calli from the germplasms,
b) incubating calli obtained in step a) under conditions suitable for germination of somatic embryos,
c) selecting the calli which produced higher than a predetermined tiller count and having healthy shoots,
d) incubating the calli selected in step c) under conditions that induce root formation on to the calli,
e) selecting the calli which produced healthy roots in step d),
f) planting the calli selected in step e) in to soil to produce plants from the calli,
g) obtaining floral meristematic cells from the plants grown in step f),
h) generating calli from the floral meristematic cells obtained in step g),
i) transforming the cells from the calli generated in step h) according to the transformation method of claim 1, and
j) selecting the calli which exhibit higher rate of transformation compared to the calli tested in step i).

16. The method of claim 1, wherein the germplasms are seeds.

17. The method of claim 1, wherein the incubations in steps a) and h) are performed on a callus induction medium.

18. The method of claim 1, wherein the incubations in steps b) and d) are performed on Murashige and Skoog basal medium (MSB) medium.
